(19) United States
(12) Patent Application Publication
(10) Pub. No.: US 2005/0186182 A1
(43) Pub. Date: Aug. 25, 2005

(54) Methods of using G-CSF mobilized C-Kit+ cells in the production of embryoid body-like cell clusters for tissue repair and in the treatment of cardiac myopathy

(76) Inventors: Theresa Deisher, Seattle, WA (US); Xiaozhen Wang, Issaquah, WA (US); C. Glenn Begley, Westlake Village, CA (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/985,835

(22) Filed: Nov. 10, 2004

Related U.S. Application Data
(60) Provisional application No. 60/518,764, filed on Nov. 10, 2003.

(51) Int. Cl. A61K 45/00; C12N 5/08

(52) U.S. Cl. 424/93.7; 435/372

(57) ABSTRACT

The present invention relates to methods of using granulocyte colony stimulating factor (G-CSF) polypeptide, alone and in conjunction with stromal cell derived factor (SDF-1) polypeptide, to increase the mobilization of c-Kit+ stem cells in the blood, bone marrow, tissue, heart or other organs for the subsequent production of embryoid body-like cell clusters. These embryoid body-like cell clusters can be used for cell replacement therapy, for the treatment of cardiac myopathy and other diseases and disorders, and for screening agents that drive or inhibit differentiation and proliferation.

### RELATED APPLICATIONS

This application claims benefit of U.S. Provisional Patent Application Ser. No. 60/518,764 filed Nov. 10, 2003, which is incorporated herein by reference in its entirety.

### FIELD OF THE INVENTION

The present invention relates to the use of granulocyte colony stimulating factor (G-CSF) polypeptide, alone and in conjunction with stromal cell derived factor-1 (SDF-1) polypeptide, to increase the mobilization of c-Kit-+ cells in the blood, bone marrow, tissue, heart or other organ. More particularly, the invention provides methods of using isolated c-Kit+ cardiac cells for the production of embryoid body-like cell clusters (EBLC), which can be used for cell replacement therapy, for the treatment of cardiac myopathy, and for screening agents that drive differentiation and proliferation.

### BACKGROUND OF THE INVENTION

Adult tissue-specific stem cells are present in various tissues and are important for the maintenance of tissues within an organ due to normal physiological processes. It remains uncertain how tissue-specific stem cells might differentiate into mature cell types in another tissue. Hematopoietic stem cells, such as bone marrow stem cells (BMSC), however, have been shown recently to possess “developmental plasticity,” the ability to differentiate into a cell of a different lineage (Korbling, N. Engl. J. Med. 349:570-582, 2003). Like embryonic stem cells (ESC), which have not differentiated, the differentiation potential of BMSC may constitute a new form of cellular therapy.

There has been growing interest in the use of BMSC as a source of cells for new therapeutic strategies, such as cell transplantation and tissue engineering. Additionally, undifferentiated BMSC may have the ability to be used to screen compounds for their proliferative, differentiating, and cytotoxic effects. Moreover, the identification of growth factors that selectively activate BMSC proliferation and differentiation may lead to the recognition of novel therapies. The use of BMSC for research and therapeutic purposes offers many of the same benefits provided by ESC without the controversy.

ESC contain all the information required to form virtually all cell types/tissues in the body. Scientists have studied mouse ESC for years and have been perfecting methods to maintain these cells in culture and control their growth and differentiation. ESC can be maintained in culture in an undifferentiated state or be allowed to differentiate. When ESC are allowed to differentiate in a suspension culture, they form spherical multicellular aggregates, or embryoid bodies (EB), that contain a variety of cell populations (Robertson, “Embryo-derived stem cells,” In: Robertson E J, ed. Teratocarcinomas and embryonic stem cells: a practical approach., First Edition. Washington, D.C.: IRL Press, pp. 71-112, 1987; Keller, Curr. Opin. Cell Biol. 7:862-869, 1995). EB, like ESC, can be useful in a variety of therapeutic and screening strategies.

The mechanisms by which circulating stem cells are recruited into various tissues for differentiation are not understood. It has been proposed that changes in the microenvironment of injured tissue may play a role in stem cell recruitment (Korbling, supra), because in the absence of tissue damage, most cells disappeared or did not integrate into the host organ (Nadal-Ginard et al., Circ. Res. 92:139-150, 2003). Several studies have demonstrated that cytokine mobilization of circulating human stem cells into the blood contributes to the generation of nonhematopoeitic cells and differentiation. For example, granulocyte-macrophage colony-stimulating factor (GM-CSF) and G-CSF have been shown to contribute to ocular neovascularization (Takahashi et al., Nat. Med. 5:434-438, 1999) and neovascularization of ischemic myocardium (Kocher et al., Nat. Med. 7:430-436, 2001). Likewise, G-CSF-mobilized BMSC were reported to generate functioning cardiomyocytes and repair the infarcted heart (Orlic et al., Proc. Natl. Acad. Sci. USA 98:10344-10349, 2001). Orlic et al. also showed c-Kit+ BMSC, injected into the contracting myocardium, differentiated into myocytes and coronary vessels and improved the function of the injured heart (Nature 410:701-705, 2001).

The administration of G-CSF and SCF has been shown to mobilize pluripotent BMSC from the bone marrow and greatly increase their number in the peripheral circulation (Orlic et al., Proc. Natl. Acad. Sci. USA, 98:10344-10349, 2001). Moreover, BMSC, enriched for the expression of c-Kit, the receptor for SCF, have been shown to induce myocardial regeneration; the regenerated myocardium contracted synchronously with the ventricle and improved cardiac function (Orlic et al., Nature 410:701-705, 2001). Although the c-Kit-+ cells comprised the majority of the BMSC, the precise identity of the specific BMSC that contributed to this regeneration in the heart has yet to be established (Nadal-Ginard et al., supra).

G-CSF causes an increase in the release of hematopoietic stem cells into the blood, and plays a role in the proliferation, differentiation, and survival of myeloid progenitor cells (Takano et al., Curr. Pharm. Des. 9:1121-1127, 2003). G-CSF and other hematopoietic growth factors including interleukin-3 (IL-3), IL-6, granulocyte-macrophage colony stimulating factor (GM-CSF), and stem cell factor (SCF) have all been reported to be positive regulators of granulopoiesis, the production of granulocytes in the bone marrow (Takano et al., Curr. Pharm. Des. 9:1121-1127, 2003). G-CSF is species cross-reactive, such that when human G-CSF is administered to another mammal such as a mouse, canine, or monkey, sustained neutrophil leukocytosis is elicited (Moore et al., Proc. Natl. Acad. Sci. USA 84:7134-7138, 1987).

C-kit, also known as stem cell factor (SCF) receptor or CD117, is a transmembrane receptor with tyrosine kinase activity (type III tyrosine kinase receptor). SCF is produced by BMSC and is expressed on both primitive and mature hematopoietic progenitor cells. SCF and c-Kit are essential for haemopoiesis, melanogenesis, and fertility. The interaction of SCF with c-Kit rapidly induces receptor dimerization and increases in autophosphorylation of tyrosine residues of the cytoplasmic domain, which in turn activates signal transduction pathways common to many growth factor receptors.

Cardiac myocytes are continuously lost and replaced throughout life (Anversa et al., Circ. Res. 83:1-14, 1998; Soonpaa et al., Circ. Res. 83:15-26, 1998). There is a small and continuously renewed subpopulation of myocytes produced by the differentiation of cardiac-derived stem cells (CSC) (Nadal-Ginard et al., supra). Undifferentiated cells, expressing antigens commonly found in bone marrow progenitor cells, like c-Kit, are also present in the atria and ventricle of the human and rodent heart (Anversa et al., Nature 415:240-243, 2002; Quaini et al., N. Engl. J. Med. 346:5-15, 2003; Nadal-Ginard et al., supra). To determine whether the c-Kit+ cells in the heart behaved like true CSC, undifferentiated Lin-c-Kit+ cells were isolated from the ventricle of adult rats, plated, cultured, and cloned [Beltrami et al., Circulation 104 (suppl II):324 (abstract), 2001; Beltrami et al. Cell 114:763-776, 2003]. The progeny of single cells expressed markers of the three main cardiac cell types: 1) myocytes, 2) smooth muscle cells, and 3) endothelial cells. The presence of these CSC in the heart suggests that there is a cycling population of cells within the heart that are able to differentiate depending upon the physiological need of the heart.

Although there is a small population of CSC, evidence suggests that adult cardiomyocytes can be also replaced by progenitor cells in bone marrow known as mesenchymal stem cells (MSC) (Deisher, IDrugs 3:649-653, 2000). MSC are capable of differentiating into a variety of tissues and their differentiation into bone, cartilage, fat, stroma, and muscle has been reported (Deisher, supra). Moreover, unfractionated marrow transplanted into adult mice resulted in the appearance of donor-derived cardiomyocytes (Bittner et al., Anat. Embryol. 199:391-396, 1999). Although evidence suggests that MSC can generate cardiomyocytes, it has yet to be determined if MSC are actually present within the heart.

Consequently, primitive cells with stem cell properties are present in the heart, either as a resident population of stem cells or as a blood-born population of stem cells that can continuously seed the tissue (Nadal-Ginard et al., supra). Understanding the regulation of myocyte formation from either BMSC or CSC provides new means for therapeutic intervention and tissue regeneration.

Accordingly, BMSC, ESC, and EB all are useful tools for in the study of differentiation, proliferation, and tissue regeneration. Thus, there is a need in the art for new methodologies to develop BMSC, ESC, and EB that can be useful for these purposes. For that reason, an object of the present invention is to provide new methods for producing and using embryoid body-like cell clusters (EBLC) and embryoid bodies (EB), which are discussed in further detail herein.

### SUMMARY OF THE INVENTION

The present invention contemplates a new method for obtaining clusters of quasi-totipotent cells from an adult for use in cell replacement therapy and drug screening. More specifically, the present invention relates the use of granulocyte colony stimulating factor (G-CSF) polypeptide, alone and in conjunction with stromal cell derived factor-1 (SDF-1) polypeptide or other agents, to increase the mobilization of c-Kit+ stem cells in the blood, bone marrow, tissue, heart or other organs. More specifically, the invention provides methods useful in isolating c-Kit+ stem cells for the production of embryoid body-like cell clusters (EBLC), which can be used for cell replacement therapy, for the treatment of cardiac myopathy and other diseases and disorders, and for screening agents that drive or inhibit differentiation and proliferation.

In one aspect, therefore, the invention provides methods of producing embryoid body-like cell clusters (EBLC) derived from c-Kit+ cells from a mammal, wherein the method comprises: administering to a mammal a composition comprising an effective amount of granulocyte colony stimulating factor (G-CSF) polypeptide to mobilize c-Kit+ cells; isolating the mobilized c-Kit-+ cells from the blood, bone marrow, tissue, heart or other organ; and culturing the c-Kit-+ cells in long-term culture medium for the development of the EBLC.

In another aspect, the invention contemplates improved methods of cell replacement therapy, the improvement comprising: administering to a mammal a composition comprising an effective amount of granulocyte colony stimulating factor (G-CSF) polypeptide to mobilize c-Kit-+ cells; isolating the mobilized c-Kit-+ cells from the blood, bone marrow, tissue, heart or other organ; culturing the c-Kit-+ cells in long-term culture medium for the development of the embryoid body-like cell clusters (ELBC); and administering the embryoid body-like cell clusters into a mammal for cell replacement therapy. Such cell replacement therapy may be used in the treatment of various diseases or disorders. Some possible diseases or disorders which may be treated with the methods of the invention include, but are not limited to, cardiac myopathies, skeletal myopathies, neural degenerative diseases, stroke, bone loss, vascular degeneration, and joint degeneration.

In a further aspect, the invention contemplates methods for screening agents for their ability to promote cell differentiation, the improvement comprising: administering to a mammal a composition comprising an effective amount of granulocyte colony stimulating factor (G-CSF) polypeptide to mobilize c-Kit-+ cells; isolating the mobilized c-Kit-+ cells from the blood, bone marrow, tissue, heart or other organ; culturing the c-Kit-+ cells in long-term culture medium for the development of the embryoid body-like cell clusters; treating the embryoid body-like cell clusters with agents that promote cell differentiation; and examining the embryoid body-like cell clusters for changes in cellular phenotype. Such cellular phenotypes include, but are not limited to, cardiomyocytes, endothelial cells, osteoblasts, chondrocytes, neurons, oligodendrocytes, adipocytes, smooth muscle cells, hematopoietic cells, hepatocytes, fibroblasts, renal cells, and germ cells. Some agents used to promote differentiation may be derived from a chemical, protein, or other library.

In yet another aspect, the invention contemplates methods for screening agents for toxicity, the improvement comprising: administering to a mammal a composition comprising an effective amount of granulocyte colony stimulating factor (G-CSF) polypeptide to mobilize c-Kit-+ cells; isolating the mobilized c-Kit-+ cells from the blood, bone marrow, tissue, heart or other organ; culturing the c-Kit-+ cells in long-term culture medium for the development of the embryoid body-like cell clusters; treating the embryoid body-like cell clusters with agents that affect cell differentiation or inhibit proliferation; and examining the embryoid body-like cell clusters for changes in cellular phenotype.

The invention also contemplates that the composition used in the methods of the invention may include at least one additional growth factor or agent. In one aspect, one of the growth factors is stromal derived growth factor-1 (SDF-1).

The invention further contemplates methods for producing embryoid body-like cell clusters derived from c-Kit-+ cells from a mammal, wherein the method comprises: isolating the c-Kit-+ cells from the blood, bone marrow, tissue, heart or other organ; and culturing the c-Kit-+ cells in long-term culture medium for the development of the embryoid body-like cell clusters.

In another aspect, the invention provides improved methods of cell replacement therapy, the improvement comprising: isolating c-Kit-+ cells from the blood, bone marrow, tissue, heart or other organ; culturing the c-Kit-+ cells in long-term culture medium for the development of the embryoid body-like cell clusters; and administering the embryoid body-like cell clusters into a mammal for cell replacement therapy. Such cell replacement therapy may be useful in the treatment of various diseases or disorders. Some possible diseases or disorders which may be treated with the methods of the invention include, but are not limited to, cardiac myopathies, skeletal myopathies, neural degenerative diseases, stroke, bone loss, vascular degeneration, and joint degeneration.

In still another aspect, the invention provides improved methods for screening agents for their ability to promote cell differentiation, the improvement comprising: isolating c-Kit-+ cells from the blood, bone marrow, tissue, heart or other organ; culturing the c-Kit-+ cells in long-term culture medium for the development of the embryoid body-like cell clusters; treating the embryoid body-like cell clusters with agents; and examining the embryoid body-like cell clusters for changes in cellular phenotype. Such cellular phenotypes include, but are not limited to, cardiomyocytes, endothelial cells, osteoblasts, chondrocytes, neurons, oligodendrocytes, adipocytes, smooth muscle cells, hematopoietic cells, hepatocytes, fibroblasts, renal cells, and germ cells. Some agents used to promote differentiation may be derived from a chemical, protein, or other library.

The invention also provides improved methods for screening agents for toxicity, the improvement comprising: isolating c-Kit-+ cells from the blood, bone marrow, tissue, heart or other organ; culturing the c-Kit-+ cells in long-term culture medium for the development of the embryoid body-like cell clusters; treating the embryoid body-like cell clusters with agents that affect cell differentiation and proliferation; and examining the embryoid body-like cell clusters for changes in cellular phenotype or cell number.

In another aspect, the invention provides improved methods for producing embryoid body-like cell clusters derived from umbilical cord blood stem cells from a mammal. Such a method generally comprises: isolating mononuclear cells from umbilical cord blood; removing non-stem cells from the mononuclear cells; and culturing the remaining mononuclear cells in long-term culture medium for the development of the embryoid body-like cell clusters. Such a method may also be used to produce embryoid body-like cell clusters from placental stem cells from a mammal. Such embryoid body-like cell clusters may be useful in cell replacement therapy; in screening agents for their ability to promote or inhibit stem cell proliferation; and in screening agents for their ability to promote or inhibit stem cell differentiation. Such cell differentiation may be detected by the observation of changes in cellular phenotype. For example, cardiocyte differentiation may be detected by the expression of a cardiocyte differentiation marker.

The invention also contemplates methods for producing cardiac progenitor cells derived from umbilical cord blood stem cells from a mammal. Such a method generally comprises: isolating mononuclear cells from umbilical cord blood; removing non-stem cells from the mononuclear cells; culturing the remaining mononuclear cells in long-term culture medium for the development of embryoid body-like cell clusters; co-culturing the embryoid body-like cell cluster with irradiated myocytes or other agent which promotes cardiac progenitor cell differentiation; analyzing the embryoid body-like cells for the presence of cardiocyte differentiation markers; and selecting embryoid body-like cells of a cardiocyte phenotype. Such cells may be useful in cardiac cell replacement therapy.

In another aspect of the methods of invention, the removal of non-stem cells is carried out by refrigerating the cells to destroy the non-stem cells. In a different aspect, the removal of non-stem cells is carried out by selecting c-Kit-+ cells (stem cells) and washing away the non-stem cells. Such a method of c-Kit-+ cell selection generally may be carried out by using a fluorescence activated cell sorter (FACS) or magnetic cell sorting (MACS).

The term “G-CSF polypeptide” or “G-CSF” as used herein is defined as naturally occurring human and heterologous species G-CSF, recombinantly produced G-CSF that is the expression product consisting of either 174 or 177 amino acids, or fragments, analogs, variants, or derivatives thereof as reported, for example in Kuga et al., Biochem. Biophys. Res. Comm. 159: 103-111 (1989); Lu et al., Arch. Biochem. Biophys. 268: 81-92 (1989); U.S. Pat. Nos. 4,810,643, 4,904,584, 5,104,651, 5,194,592, 5,214,132, 5,218,092, 5,362,853, 5,416,195, 5,606,024, 5,681,720, 5,714,581, 5,773,581, 5,795,968, 5,824,778, 5,824,784, 5,939,280, 5,994,518, 6,017,876, 6,027,720, 6,166,183, and 6,261,550; U.S. Pat. Appl. No. US 2003/0064922; EP 0 335423; EP 0 272703; EP 0 459630; EP 0 256843; EP 0 243153; WO 9102874; Australian Application document Nos. AU-A-10948/92 and AU-A-76380/91. Included are chemically modified G-CSFs, see, e.g., those reported in WO 9012874, EP 0 401384 and EP 0 335423. See also, WO 03006501; WO 03030821; WO 0151510; WO 9611953; WO 9521629; WO 9420069; WO 9315211; WO 9305169; JP 04164098; WO 9206116; WO 9204455; EP 0 473268; EP 0 456200; WO 9111520; WO 9105798; WO 9006952; WO 8910932; WO 8905824; WO 9118911; and EP 0 370205. Also encompassed herein are all forms of G-CSF, such as AlbugraninTM, NeulastaTM®, Neupogen®, and Granocyte®.

The term “long term culture medium” or “LTC medium” as defined herein may include any media containing minimally essential nutrients capable of maintaining viable cells over long term culture. LTC medium as used herein is Iscove's Modified Dulbecco's Medium (IMDM) plus horse serum (10%) and/or bovine serum (10%).

The term “embryoid body-like cell cluster” or “EBLC” as used herein is defined as a mass of c-Kit-+ cells which aggregate together upon long term culture in the absence of appropriate adherent feeder layers to form cystic structures morphologically indistinguishable from embryoid bodies, but not derived from embryonic stem cells. EBLC typically form within two weeks in culture; however, they can be maintained in culture for long periods of time. The terms “embryoid body-like cell clusters” and “embryoid body-like cells” are used interchangeably herein.

The term “embryoid body” or “EB” as used herein is defined as a mass of embryonic stem cells.

The term “differentiation” as used herein is defined as the sum of the processes whereby apparently uncommitted cells develop and change to attain the cellular phenotype of their adult form and function.

### DETAILED DESCRIPTION OF THE INVENTION

G-CSF has been found to be useful alone, or in combination with stromal cell derived factor-1 (SDF-1), in the mobilization of bone marrow stem cells. The present invention addresses a role for G-CSF polypeptide, alone and in combination with other agents or growth factors such as, but not limited to SDF-1, in the increased production and mobilization of c-Kit+ stem cells into the blood, bone marrow, tissue, heart or other organs.

The present invention also contemplates methods of isolating and culturing these c-Kit+ stem cells for use in the treatment of various diseases and disorders. More specifically, the invention contemplates methods for using these cells in the treatment of cardiomyopathy to enhance cardiac contractility, limit changes in chamber dimensions, and improve cardiac output.

Discussed in further detail herein below are the use of G-CSF in the mobilization of c-Kit+ stem cells, methods of administering G-CSF to mobilize c-Kit+stem cells, pharmaceutical compositions comprising G-CSF, methods of using c-Kit+ stem cells for tissue repair or regeneration, methods of using embryonic stem cells in drug and toxicity screening, and methods of using differentiation markers to study stem cell differentiation.

The section headings are used herein for organizational purposes only, and are not to be construed as in any way limiting the subject matter described.

A. Use of G-CSF in the Mobilization of C-Kit+ Stem Cells

In one embodiment, the present invention contemplates methods of using G-CSF to increase the number of c-Kit+ bone marrow-derived stem cells (BMSC) and cardiac-derived stem cells (CSC), and isolating these cells from the blood, bone marrow, tissue, heart or other organ to form embryonic-like bodies (EBLC) in culture. The methods of the present invention exploit the use of G-CSF polypeptide, in conjunction with stromal derived factor-1 (SDF-1), in the mobilization of stem cells.

The present invention also contemplates methods of using other agents such as, but not limited to, stem cell factor (SCF or c-Kit ligand) in conjunction with G-CSF polypeptide for the mobilization of c-Kit+ stem cells into the blood, bone marrow, tissue, heart or other organ. SCF is an essential hematopoietic cytokine, which interacts with other cytokines to promote viability of stem cells and facilitate their differentiation, proliferation, adhesion, and functional activation.

The term “stem cell factor” or “SCF” as used herein refers to naturally-occurring SCF (e.g. natural human-SCF) as well as non-naturally occurring (i.e., different from naturally occurring) polypeptides having amino acid sequences and glycosylation sufficiently duplicative of that of naturally-occurring stem cell factor to allow possession of a hematopoietic biological activity of naturally-occurring stem cell factor. The term “SCF” as used herein is also defined as recombinantly produced SCF, or fragments, analogs, variants, or derivatives thereof as reported, for example in U.S. Pat. Nos. 6,204,363, 6,207,417, 6,207,454, 6,207,802, 6,218,148, and 6,248,319. Stem cell factor has the ability to stimulate growth of early hematopoietic progenitors which are capable of maturing to erythroid, megakaryocyte, granulocyte, lymphocyte, and macrophage cells. SCF treatment of mammals results in absolute increases in hematopoietic cells of both myeloid and lymphoid lineages. One of the hallmark characteristics of stem cells is their ability to differentiate into both myeloid and lymphoid cells [Weissman, Science 241:58-62 (1988)].

SCF is produced by bone marrow stromal cells and is expressed on both primitive and mature hematopoietic progenitor cells. Within the human haemopoietic system, c-Kit protein is expressed by approximately 70% of CD34+ cells in bone marrow, as well as by megakaryocytes, mononuclear cells, and activated platelets (Ashman, Int. J. Bioch. & Cell Biol. 31:1037-1051, 1999). SCF and SCF receptor (c-Kit) are essential for haemopoiesis, melanogenesis, and fertility. The interaction of SCF with c-Kit rapidly induces receptor dimerization and increases in autophosphorylation of tyrosine residues of the cytoplasmic domain (Linnekin, Int. J. Bioch. & Cell Biol. 31:1053-1074, 1999). These phosphotyrosine residues become docking sites for various cytoplasmic signaling molecules containing SH2 domain (Boissan et al., J. Leukoc. Biol. 67:135-148, 2000). C-Kit activates signal transduction pathways common to many growth factor receptors.

Human G-CSF can be obtained and purified from a number of sources. Natural human G-CSF can be isolated from the supernatants of cultured human tumor cell lines. The development of recombinant DNA technology has enabled the production of commercial scale quantities of G-CSF in glycosylated form as a product of eukaryotic host cell expression, and of G-CSF in non-glycosylated form as a product of prokaryotic host cell expression. See, for example, U.S. Pat. No. 4,810,643 (Souza) incorporated herein by reference.

B. Methods of Administering GCSF to Mobilize Stem Cells

As mentioned herein above, it is contemplated that methods of the present invention will use G-CSF polypeptide alone and in conjunction with SDF-1 in the mobilization of c-Kit+ stem cells to the blood, bone marrow, tissue, heart or other organ. The present section provides a description of how G-CSF may be therapeutically administered in the methods of the invention.

One of the therapeutic embodiments of the present invention is the provision, to a subject in need thereof, compositions comprising G-CSF polypeptide. G-CSF polypeptide may have been generated through recombinant means or by automated peptide synthesis. The G-CSF formulations for such a therapy may be selected based on the route of administration and may include liposome and micelle formulations as well as classic pharmaceutical preparations.

G-CSF proteins are formulated into appropriate preparation and administered to one or more sites within the subject in a therapeutically effective amount. In particularly preferred embodiments, the human G-CSF protein-based therapy is effected via continuous or intermittent intravenous administration. By “effective amount” the present invention refers to that amount of human G-CSF polypeptide that is sufficient to support an observable change in the level of one or more biological activities of G-CSF. The change may be an increased level of G-CSF activity. Preferably, the change is an increase in bone marrow stem cell mobilization or circulation to the ischemic or damaged tissue resulting in diminished tissue damage or increased tissue growth.

Those of skill in the art will understand that the amounts of human G-CSF polypeptides administered for therapeutic use may vary. It is contemplated that the specific activity of the human G-CSF protein preparation may be in the range of about 100 units/mg of protein to about 500 units/mg protein. Thus, a given preparation of a human G-CSF protein may comprise about 100 units/mg protein, about 125 units/mg protein, about 150 units/mg protein, about 175 units/mg protein, about 200 units/mg protein, about 225 units/mg protein, about 250 units/mg protein, about 275 units/mg protein, about 300 units/mg protein, about 325 units/mg protein, about 350 units/mg protein, about 375 units/mg protein, about 400 units/mg protein, about 425 units/mg protein, about 450 units/mg protein, about 475 units/mg protein and about 500 units/mg protein. A particularly preferred range is from about 100 units/mg protein to about 200 units/mg protein; a more preferable range is between about 150 to about 200 units/mg protein. Preferably, the protein composition is substantially free of contaminating factors, contamination level of less than 0.02% (w/w). Human G-CSF compositions, suitable for injection into a patient, can be prepared, for example, by reconstitution with a pharmacologically acceptable diluent of a lyophilized sample comprising purified human G-CSF and stabilizing salts.

Administration of the compositions can be systemic or local, and may comprise a single site injection of a therapeutically-effective amount of the human G-CSF protein composition. Any route known to those of skill in the art for the administration of a therapeutic composition of the invention is contemplated including, for example, intravenous, intramuscular, subcutaneous or a catheter for long-term administration. Alternatively, it is contemplated that the therapeutic composition may be delivered to the patient at multiple sites. The multiple administrations may be rendered simultaneously or may be administered over a period of several hours. In certain cases, it may be beneficial to provide a continuous flow of the therapeutic composition. Additional therapy may be administered on a period basis, for example, daily, weekly, or monthly.

In addition to therapies based solely on the delivery of the human G-CSF, combination therapy is specifically contemplated. In the context of the present invention, it is contemplated that the human G-CSF therapy could be used similarly in conjunction with other agents such as, but not limited to, SDF-1 that may promote mobilization of c-Kit+BMSC to the circulation, heart, bone marrow, and other organs.

To achieve the appropriate therapeutic outcome, using the methods and compositions of the present invention, one would generally provide a composition comprising human G-CSF and at least one other therapeutic agent (second therapeutic agent). In the present invention, it is contemplated that the second therapeutic agent may involve the administration of SDF-1. It is also contemplated that another therapeutic agent may involve the administration or inclusion of at least one additional factor selected from the group consisting of: EPO, MGDF, SCF, GM-CSF, M-CSF, CSF-1, IL-1, IL-2, IL-3, IL-4, IL-5, IL-6, IL-7, IL-8, IL-9, IL-10, IL-11, IL-12, or other various interleukins, IGF-1, LIF, interferon (such as a, B, gamma or consensus), neurotrophic factors (such as BDNF, NT-3, CTNF or noggin), other multi-potent growth factors (such as, to the extent these are demonstrated to be such multi-potent growth factors, flt-3/flk-2 ligand, stem cell proliferation factor, and totipotent stem cell factor), fibroblast growth factors (such as FGF), human growth hormone and analogs, fusion molecules, and other derivatives of the above. For example, G-CSF in combination with SCF has been found to mobilize peripheral blood progenitor cells in vivo. Ex vivo, for example, G-CSF in combination with SCF, IL-3 and IL-6 has been found useful for expansion of peripheral blood cells. Likewise, G-CSF will provide for similar uses.

The combination therapy compositions would be provided in a combined amount effective to produce the desired therapeutic outcome in the mobilization of c-Kit+ BMSC. This process may involve contacting the cells with human G-CSF polypeptide and the second agent(s) or factor(s), such as, but not limited to SDF-1 at the same time. This may be achieved by administering a single composition or pharmacological formulation that includes both agents, or by administering two distinct compositions or formulations, at the same time, wherein one composition includes the human G-CSF therapeutic composition and the other includes the second therapeutic agent.

Alternatively, the human G-CSF treatment may precede or follow the other agent treatment by intervals ranging from minutes to weeks. In embodiments where the second therapeutic agent and the human G-CSF are administered separately, one would generally ensure that a significant period of time did not expire between the times of each delivery, such that the second agent and the human G-CSF would still be able to exert an advantageously combined effect. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

C. Pharmaceutical Compositions Comprising G-CSF

As mentioned herein above, the present invention also comprehends methods using pharmaceutical compositions comprising effective amounts of G-CSF polypeptide together with pharmaceutically acceptable diluents, preservatives, solubilizers, emulsifiers, adjuvants and/or carriers useful in G-CSF therapy. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., thimersol, benzyl alcohol), and bulking substances (e.g., lactose, mannitol); incorporation of the material into particulate preparations of polymeric compounds, such as polylactic acid, polyglycolic acid, etc., or in association with liposomes or micelles. Such compositions will influence the physical state, stability, rate of in vivo release, and rate of in vivo clearance of the G-CSF. See, e.g., Remington's Pharmaceutical Sciences, 18th Ed. (1990) Mack Publishing Co., Easton, Pa., pages 1435-1712, which are herein incorporated by reference.

Derivatives of G-CSF are also comprehended herein. Such derivatives include molecules modified by one or more water soluble polymer molecules, such as polyethylene glycol, or by the addition of polyamino acids, including fusion proteins (procedures for which are well-known in the art). Such derivatization may occur singularly at the N- or C-terminus or there may be multiple sites of derivatization. Substitution of one or more amino acids with lysine may provide additional sites for derivatization. (See U.S. Pat. No. 5,824,784 and U.S. Pat. No. 5,824,778, incorporated by reference herein).

G-CSF or derivatives thereof may be formulated for injection, or oral, nasal, pulmonary, topical, or other types of administration as one skilled in the art will recognize. The formulation may be liquid or may be solid, such as lyophilized, for reconstitution.

In order to prepare human G-CSF containing compositions for clinical use, it may be necessary to prepare the viral expression vectors, proteins, and nucleic acids as pharmaceutical compositions, i.e., in a form appropriate for in vivo applications. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the human G-CSF analog or an expression vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well-known in the art. Except insofar as any conventional media or agent is incompatible with the vectors or cells of the present invention, its use in therapeutic compositions is contemplated. Supplementary active ingredients also can be incorporated into the compositions.

The active compositions used in the methods of the present invention include classic pharmaceutical preparations. Administration of these compositions according to the present invention will be via any common route so long as the target tissue is available via that route. The pharmaceutical compositions may be introduced into the subject by any conventional method, e.g., by intravenous, intradermal, intramuscular, intramammary, intraperitoneal, intrathecal, retrobulbar, intrapulmonary (e.g., term release); by oral, sublingual, nasal, anal, vaginal, or transdermal delivery, or by surgical implantation at a particular site. The treatment may consist of a single dose or a plurality of doses over a period of time.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents (for example, sugars or sodium chloride). Prolonged absorption of the injectable compositions can be brought about by the use in the compositions of agents delaying absorption (for example, aluminum monostearate and gelatin).

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum-drying and freeze-drying techniques that yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well-known in the art. Except insofar as any conventional media or agent is incompatible with the active ingredient, its use in the therapeutic compositions is contemplated. Supplementary active ingredients also can be incorporated into the compositions.

For oral administration of the compositions used in the methods of the present invention, G-CSF may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions used in the methods of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

Generally, an effective amount of G-CSF, or derivatives thereof, will be determined by the age, weight, and condition or severity of disease of the recipient. See, Remington's Pharmaceutical Sciences, supra, pages 697-773, herein incorporated by reference. Typically, a dosage of between about 0.001 μg/kg body weight/day to about 1000 μg/kg body weight/day, may be used, but more or less, as a skilled practitioner will recognize, may be used. A preferred dosage in an adult human is approximately 300 μg/day. Dosing may be one or more times daily, or less frequently, and may be in conjunction with other compositions as described herein. It should be noted that the present invention is not limited to the dosages recited herein.

“Unit dose” is defined as a discrete amount of a therapeutic composition dispersed in a suitable carrier. For example, where polypeptides are being administered parenterally, the polypeptide compositions are generally injected in doses ranging from 1 μg/kg to 100 mg/kg body weight/day, preferably at doses ranging from 0.1 mg/kg to about 50 mg/kg body weight/day. Parenteral administration may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, supra, pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

Appropriate dosages may be ascertained through the use of established assays for determining level of BMSC mobilization in conjunction with relevant dose-response data. It will depend on the drug's specific activity, the responsiveness of the patient, the age, condition, body weight, sex and diet of the patient, the severity of any infection, time of administration and other clinical factors. As studies are conducted, further information will emerge regarding appropriate dosage levels and duration of treatment.

It will be appreciated that the pharmaceutical compositions and treatment methods of the invention may be useful in fields of human medicine and veterinary medicine. Thus the subject to be treated may be a mammal, preferably a human.

D. Methods of Using C-Kit+ Stem Cells for Tissue Repair or Regeneration

The methods of the present invention contemplate the use of embryoid body-like cell clusters (EBLC) derived from c-Kit+ stem cells, described herein, to promote tissue repair or regeneration. Therefore, the present section provides a brief summary of what is known about the role of stem cells in tissue repair or regeneration to the extent that such a summary will facilitate a better understanding of the methods of the present invention.

Primitive cells in bone marrow have the capacity, both in vitro and in vivo, to give rise to cells of all three germ layers. Stem cells of mesenchymal/stromal and hematopoietic origin have been suggested to have the potential to differentiate like ESC. However, the mechanism for this “transdifferentiation” of adult stem cells is controversial and not well understood (Orlic et al., Ann. N.Y. Acad. Sci. 996:152-157, 2003).

Stem cell replacement therapy is contemplated in the methods of the invention for tissue repair and regeneration. Tissue regeneration using BMSC is known in the art and has been demonstrated in a variety of tissues including, but not limited to, muscle (Ferrari et al., Science 279:1528-1530, 1998) and heart (Jackson et al., J. Clin. Invest. 107:1395-1402; Kocher et al., Nature Med. 7: 430-436, 2001; Orlic et al., Nature 410:701-705, 2001; and Orlic et al., Proc. Natl. Acad. Sci. USA 98:10344-10349, 2001). Orlic et al. (Nature, supra) used direct injection of BMSC into the heart three to five hours after ligation of the left coronary artery in a mouse model, resulting in the generation of new cardiomyocytes and endothelial cells in the zone of ischemic myocardium. These same researchers later reported that the proliferation of BMSC in mice, effected by the treatment of mice with G-CSF prior to affecting occlusion of the left coronary artery, could ameliorate myocardial injury induced by the occlusion (Orlic et al., Proc. Natl. Acad. Sci. USA, supra). Taken together, these studies indicate that stem cell therapy provides a novel therapeutic strategy in regenerating myocardium and treating heart disease.

The present invention provides examples of how stem cell therapy was successfully used in models of cardiomyopathy to increase cardiac contractility, limit changes in chamber dimensions, improve global cardiac output, and reduce overall mortality. The present invention also contemplates the use of the c-Kit-+ cells in stem cell therapy for tissue repair in the treatment of many other diseases and disorders.

Cell replacement therapy in the methods of the invention contemplates placing isolated c-Kit-+ cells in long term culture until the appearance of embryoid body-like cell clusters (EBLC) is observed. The resulting EBLC may be administered into the patient as an explant or as single cells after dispersion by gentle trypsin or versene digestion to single cell suspensions. The single cell suspension derived from the EBLC can differentiate into all cell types, and in the appropriate in vivo milieu can be driven by local growth factors and other local cues to selectively replace or augment the specific cell types necessary for improvement of organ function in that specific patient.

E. Methods of Using Embryonic Stem Cells in Drug and Toxicity Screening

The methods of the present invention also contemplate the use of EBLC to test compounds for activity in promoting or inhibiting the proliferation and/or differentiation of said EBLC. In general, a compound being tested is contacted with a population of EBLC in the presence and/or absence of other agents known to promote or inhibit proliferation and/or differentiation of cells or tissues.

High throughput screening is contemplated in the methods of the invention. High throughput screening is the process by which multiple compounds are tested for biological activity or binding activity with various target molecules. Test compounds act to either stimulate or inhibit proliferation, differentiation, and biological activity. Likewise, test compounds may compete for binding of a natural ligand to its receptor, as agonists or antagonists for receptor-mediated intracellular processes, and so forth. High throughput screening seeks to screen large numbers of compounds rapidly and in parallel.

Positive high throughput screening results are usually called hits. Compounds resulting in hits are collected for further testing in which, for example, the potency of compound in inducing differentiation or stimulating cellular proliferation is determined. Hits then become lead compounds. Further synthesis is then required to provide a variety of compounds structurally related to the lead. These sublibraries are then screened against targets in order to choose optimal compounds.

Compounds to be tested include known or suspected growth factors, and analogs thereof. Such compounds can include, but are not limited to, angiogenin, bone morphogenic proteins-1-15, brain derived neurotrophic factor, ciliary neutrophic factor, cytokine-induced neutrophil chemotactic factors 1, 2 α, and 2 β, β endothelial cell growth factor, endothelin 1, epidermal growth factor, epithelial-derived neutrophil attractant, fibroblast growth factors 1-12, fibroblast growth factor acidic, fibroblast growth factor basic, granulocyte-macrophage colony-stimulating factor (GM-CSF), granulocyte-colony stimulating factor (G-CSF), growth related proteins, heparin binding epidermal growth factor, hepatocyte growth factor, hepatocyte growth factor, insulin-like growth factors, insulin-like growth factor binding proteins, keratinocyte growth factors, leukemia inhibitory factor, nerve growth factors, neurotrophins, placenta growth factors, platelet-derived endothelial cell growth factor, platelet derived growth factors, pre-B cell growth stimulating factor, stem cell factor, stromal cell derived factor-1 (SDF-1), transforming growth factors, latent transforming growth factor, transforming growth factor β binding proteins, tumor necrosis factors, urokinase-type plasminogen activator, vascular endothelial growth factor, and chimeric proteins and biologically or immunologically active fragments thereof.

Other compounds to be tested may include known or suspected cytotoxic or embryotoxic agents. These compounds may be tested alone or with additional factors to determine their toxic effect on the EBLC.

Additional compounds to be tested include libraries and combinatorial libraries of natural compounds not known previously to have an effect on proliferation or differentiation. Such compounds can be administered alone or in conjunction with other compounds. Such compounds are screened for proliferating activity by administering them to the embryoid body-like cell clusters in growth media, and measuring an increase in cell number, or incorporation of ³H-thymidine into the cells. Compounds are screened for promoting differentiation by administering them to the embryoid body-like cell clusters in growth media, and monitoring changes in morphological appearance or the presence or absence of differentiation markers as set out below. More particularly, compounds can be monitored for activity in promoting differentiation into cells such as, but not limited to, cardiocytes, smooth muscle cells, skeletal muscle cells, osteoblasts, chondrocytes, neurocytes, hepatocytes, renal cells, germ cells, and adipocytes. Likewise, differentiation promoting activity is detected by determining changes in morphological appearance, as well as detection of one of the differentiation markers characteristic of the differentiated cell type.

Compounds with activity in promoting differentiation into one of the above cell types are useful in treating patients with degenerative diseases of the circulatory system, muscular system, skeletal system, connective tissue system, nervous system, liver, kidney, and metabolic system. Compounds with activity in inhibiting differentiation of embryoid body-like cell clusters into certain cell types, such as, but not limited to, adipocytes can also be useful. For example, compounds that inhibit differentiation into adipocytes can be used in the treatment of obesity. Such compounds are screened for inhibiting differentiation by contacting them with the embryoid body-like cell clusters in conditions that would otherwise lead to differentiation, and monitoring a decreased expression in differentiation marker or decrease in extent of differentiation in morphological appearance.

Compounds identified as promoting or inhibiting growth or differentiation by such screening with the EBLC of the invention are formulated for therapeutic use as pharmaceutical compositions as set out below.

The present invention further contemplates the use of ESC tests known in the art in screening compounds for their effects on the proliferation and differentiation of the EBLC of the invention. Over the last 20 years, scientists have been working on mouse ESC and have been perfecting methods to maintain these cells in culture and control their growth and differentiation. Key findings include the ability to maintain these cells in an undifferentiated, stem cell state in vitro for indefinite periods of time by stimulating them with the cytokine leukemia inhibitory factor (LIF). ESC “spontaneously” differentiate upon withdrawal of LIF to form various cell types, via the intermediate formation of cell clusters or spherical multicellular aggregates called embryoid bodies (EB).

EB have been shown to contain a variety of cell populations (Robertson, supra; Keller, supra). By varying the culture conditions in which EB are maintained, it has been possible to generate cultures enriched with a particular cell type including, neurons, adipocytes, myocytes, and blood cells. EB are an interesting model to study differentiation and proliferation. Likewise, they can be useful in studying cytotoxic effects of different compounds. The present invention contemplates the application of known culturing methods for ESC with the EBLC of the invention. Like ESC, EBLC possess the ability to differentiate into multiple lineages, and therefore are promising sources for new therapeutic strategies such as cell transplantation and tissue engineering.

The present invention contemplates the use of various growth factors and agents to direct differentiation of EBLC. Some success has been achieved in the art with directing the differentiation of mouse ESC. ESC can differentiate into different embryonic cells/tissues depending on culture conditions. ESC have been induced to differentiate into cardiomyocytes (Klug et al., J Clin Invest 98:216-224, 1996), smooth muscle cells (Drab et al., FASEB J 11:905-915, 1997), hepatocytes (Yamada et al., Stem Cells 20:146-154, 2002), and neuronal cells (Lee et al., Nat. Biotechnol. 18:675-679, 2000).

The invention also contemplates methods of using EBLC in testing the in vitro cytotoxicity or embryotoxicity of compounds by the use of various assays. This technology has been used on ESC and can be applied to the use of EBLC as well. For example, Laschinski et al. (Repro. Toxicol. 5:57-64, 1991) published an assay for measuring the embryotoxic potential of teratogenic agents in mouse ESC (Laschinski et al., supra). Spielmann et al. (In Vitro Toxicol. 10: 119-127, 1997) also published an assay, called the embryonic stem cell test (EST), which takes advantage of the potential of ESC to differentiate in culture, to determine embryotoxicity. The test measures the inhibition of differentiation and differences in sensitivity between embryonic and adult tissues to cytotoxic damage. The test was developed only after it was found that ESC can be maintained in the undifferentiated stage in the presence of the cytokine leukemia inhibiting factor (LIF). The inhibition of differentiation of ESC and the inhibition of growth of ESC and 3T3 cells are the three selected endpoints in the EST for predicting the embryotoxic potential of chemicals. The embryotoxicity of a compound can also be measured by using the embryotoxicity screening methods of Scholz et al. (Cells Tissues Organs 165:203-211, 1999). Thus, it is apparent that many tests are available and are known to one of skill in the art for screening toxicity in ESC and EB and can be used in the methods of the present invention.

F. Methods of Using Differentiation Markers in Screening

Methods of the invention contemplate the use of various differentiation markers to study the effects of growth factors or agents on the differentation of the c-Kit+ BMSC or CSC. Differentiation can be recognized by changes in morphological appearance of the cells and detection of the presence of various differentiation markers. Differentiation of EBLC and EB into various cell types can be recognized by characteristic appearances using light and electron microscopy. For example, the differentiation of said structures into cardiocytes can be recognized under light microscopy by the cells' bifurcated appearance, junctional complexes, sarcomeres, and ability to contract. Likewise, cardiocytes can be recognized by their ability to form an electric potential across confluent cells and transmit signal across the cells.

Differentiation into cardiocytes can be detected with markers such as, but not limited to, connexin 43, alpha-sarcomeric actin, cardiac myosin heavy chain beta or alpha, SERCA2, and the transcription factors MEF2C or GATA4. Differentiation into smooth muscle cells can be detected with markers such as, but not limited to, smooth muscle α-actin. Differentiation into skeletal muscle cells can be detected with markers such as, but not limited to, myosin isozyme expression or under light microscopy with a muscle-specific pattern of creatine kinase isozyme expression. Differentiation into osteoblasts can be detected with markers such as, but not limited to, alkaline phosphatase (ALP), osteocalcin, parathyroid hormone (PTH)-induced cAMP expression. Differentiation into chondrocytes can be detected with markers such as, but not limited to, type II cartilage, aggrecan, collagen type IB, and Alcian Blue, which detects production of chondroitin sulfate. Differentiation into neurocytes can be detected with markers such as, but not limited to, synaptophysin, chromogranin, neuron-specific enolase (NSE), class II beta tubulin, MAP-2, tau protein, neurofilament protein (NFP), nestin, and the neuron specific adhesion molecules. Differentiation into hepatocytes can be detected with markers such as, but not limited to, albumin 1(Alb-1), hepatocyte nuclear factor 3, glutathione S-transferase (GST), M2-pyruvate kinase (M2-PK), and cellular uptake of indocyanine green (ICG). Differentiation into renal cells can be detected with markers such as, but not limited to nephrin. Differentiation into sperm cells and oocytes can be detected with markers such as, but not limited to, phospolipase C zeta, sperad, and H1oo. Differentiation into adipocytes can be detected with markers that detect the presence of lipids such as, but not limited to, Oil Red 0 staining.

### G. EXAMPLES

The present invention is described in more detail with reference to the following non-limiting examples, which are offered to more fully illustrate the invention, but are not to be construed as limiting the scope thereof. The examples illustrate the effect of G-CSF and SDF-1 on c-Kit-+ cell isolation and the formation of embryoid body-like cell clusters (EBLC); the role of c-Kit-+ cells in tissue repair in an adriamycin-induced model of cardiomyopathy; the role of c-Kit-+ cells in tissue repair in a ligation-induced model of ischemic cardiomyopathy; the use of EBLC in a high throughput screening assay; the use of EBLC in the treatment of cardiomyopathy and other diseases; the isolation of cardiac c-Kit-+ cells from mice; the formation of EBLC from human umbilical cord blood progenitor cells; the differentiation of human umbilical cord blood progenitor cells into cardiac-specific cells; and the formation of EBLC from placental progenitor cells. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods that are disclosed and still obtain a like or similar result without departing from the spirit and scope of the invention.

### Example 1

### The Effect of G-CSF and SDF-1 on C-Kit+ Cell Isolation and the Formation of Embryoid Body-Like Cell Clusters in Culture

To determine the proliferative effect of G-CSF, alone and in conjunction with SDF-1, on the number of c-Kit-+ cardiac cells in the heart and bone marrow, experiments were performed as set out below. C-Kit-+ cells were isolated from the hearts, and from femur and sternum bone marrow of 8-10 week old C57B16/J mice. For isolation of cells from the femur and sternum, the bones were flushed with 1 ml PBS+fetal bovine serum (FBS, 2%). Red blood cells were lysed. Lineage negative (Lin−) cells were isolated using a lineage antibody cocktail containing hiotinylated anti-CD3, anti-GR-1, anti-CD45R, Anti-Ter119 and anti-CD11b, and anti-biotin magnetic beads. The lineage positive cells were retained on a magnetic cell sorting (MACS) column, and flow-through Lin-cells were positively selected for c-Kit-+ cells by MACS using anti-c-Kit-biotin and anti-biotin magnetic beads.

For isolation of cells from the heart, mice were perfused under anesthesia to remove all blood from the heart. Ventricular tissue was removed, minced into small pieces and digested in Hank's Buffered Salt Solution with FBS (2%), collagenase (200U/ml), hyaluronidase (300U/ml), DNase I (50U/ml), and dispase (1U/ml) for 30 minutes at 37° C. with agitation. Tissue was further digested with versene at room temperature for 10 minutes, and passed serially through a 70 micron filter and then a 30 micron filter. C-Kit-+ cells were sorted and selected using MACS with anti-c-Kit-FITC and anti-FITC magnetic beads.

In vitro culture of isolated c-Kit-+ cells in long term culture (LTC) medium [LTC=Iscove's Modified Dulbecco's Medium (IMDM) plus horse serum (10%) and/or bovine serum (10%)] resulted in the appearance of embryoid body-like cell clusters (EBLC) within 2 to 3 weeks. A subset of the EBLC ultimately showed beating foci of cardiomyocytes within the body, reminiscent of embryonic stem cell (ESC) cultivation as embryoid bodies in hanging drops. Another subset gave rise to germ cell-like appearing structures.

Original isolates of cardiac c-Kit-+ cells from the heart have been cultured for over nine months, and have been repeatedly induced to give rise to EBLC clumps and the other phenotypes originally noted. An additional isolation of cells (over three months ago) has yielded the same phenotype of cell clumps and clusters as the original isolation of c-Kit-+ cells (over nine months ago).

In vivo treatment of mice with G-CSF polypeptide (500 μg/kg, administered subcutaneously via daily injections on days one through six) increased the number of c-Kit-+ cells that could be isolated from the heart on day 6 (from 135.7/million cells to 2954.4/million cells) and increased the number of circulating neutrophils (from 0.415×10³ cells to 6×10³ cells/ml blood). In vivo treatment of mice with SDF-1 (20 ng, administered intrapericardially (IPC) via daily injections on days two through six), in conjunction with G-CSF treatment, potentiated the effect of G-CSF on the number of c-Kit-+ cells isolated from the heart without affecting the number of circulating neutrophils. In vivo treatment of mice with Flt3 ligand, a known mobilizer of hematopoietic stem cells from the marrow, did not have an effect on cardiac c-Kit-+ cell number, indicating that the increase in cardiac c-Kit-+ cells did not result from mobilization and homing of the prototypical hematopoietic stem cell from the marrow. Furthermore, Flt3 ligand effectively increased the number of circulating monocytes, as well as neutrophils, indicating that the increase in cardiac c-Kit+ cells did not result from increases and homing of monocytes to the heart, as might be suggested based on the work of Zhao et al. (Proc. Natl. Acad. Sci. USA 100:2426-2431, 2003). G-CSF mobilized c-Kit-+ cells to the heart, and this effect was enhanced by the local administration of SDF-1. (See Table 1.)

TABLE 1
Effect of growth factor treatment on cell number. All numbers are
presented as fold increases in cell number after treatment.

| Fold Increase | | | | Cardiac |
|---|---|---|---|---|
| in Cell Number | | | CFU- | c-Kit+ |
| with Treatment | Monocytes/ | C-Kit+ cells/ | GEMM/ | cells (total |
| vs. Vehicle | μL blood | μL blood | μL blood | extractable) |
| SDF-1 | 1.5 | 1.3 | 1 | 1.5 |
| G-CSF | 0.7 | 2.5 | 5.5 | 1.7 |
| G-CSF + SDF-1 | 1.51 | 2.4 | 11.4 | 2.8 |
| Flt3L | 5.6 | 55.2 | 411.7 | 0.5 |
| Flt3L + SDF-1 | 36.7 | 59.9 | 151.1 | 1.7 |

In vitro culture of the cardiac c-Kit-+ cells from mice treated in vivo with G-CSF differed from the culture of cardiac c-Kit-+ cells from naïve or saline-treated mice in that the cells expanded as single cells in culture to a greater extent and the formation of EBLC was delayed for at least two weeks compared to c-Kit-+ cells from saline or naïve-treated mice. These results suggest that in vivo treatment with G-CSF either induced commitment of the cells to a renewing progenitor cell, less primitive than the cells which give rise to EBLC, or that the G-CSF treatment maintained the in vivo c-Kit-+ cells in a less differentiated state than in non-treated mice.

The present invention also contemplates the isolation of c-Kit+ cells from the blood, bone marrow, tissue, heart or other organ. For isolation of cells from the blood, blood is drawn from the patient and cells are precipitated via centrifugation. Red blood cells are lysed and lineage negative (Lin−) cells are isolated using a lineage antibody cocktail containing biotinylated anti-CD3, anti-GR-1, anti-CD45R, Anti-Ter119 and anti-CD11b, and anti-biotin magnetic beads. The lineage positive cells are retained on a magnetic cell sorting (MACS) column, and flow-through Lin-cells are positively selected for c-Kit-+ cells by MACS using anti-c-Kit-biotin and anti-biotin magnetic beads. C-Kit-+ cells are cultured as set out above and are subsequently used in tissue repair or in drug or toxicity screening.

### Example 2

### Role of C-Kit+ Cells in Tissue Repair in an Adriamycin-Induced Model of Cardiomyopathy

To determine the effect of cardiac c-Kit-+ cells in tissue repair, a mouse model of cardiomyopathy was used. In this model, adriamycin (doxorubicin hydrochloride) is used to induce cardiac dysfunction and ultra-structural damage to the heart.

C-Kit-+ cells were isolated from the heart or femur and sternum bone marrow of healthy mice (as set out above in Example 1), expanded in vitro for three weeks, and labeled in vitro with a tracking marker such as DiI or BrdU. Labeled c-Kit+ cells were injected either retroorbitally (RO) into the RO sinus or intrapericardially (IPC) into the pericardial sac into mice under isolflourane anesthesia at a volume of 10,000 to 1,000,000 cells per mouse, at least three days after adriamycin challenge.

Baseline echocardiograms were obtained prior to injection of adriamycin (doxorubicin hydrochloride) to determine cardiac function (measurements include fractional shortening, ejection fraction, cardiac chamber volumes, and wall thicknesses). The mice (C57B1/6, 7-8 weeks of age) were anesthetized by intraperitoneal (IP) injection of ketamine: rompun mouse cocktail; body temperature was maintained at 37° C. during the assessment; and echocardiograms were obtained non-invasively by the use of an ultrasound (SONOS 5500, Agilent). While under general anesthesia, a single RO dose of adriamcyin was administered (ranging from 0-50 mg/kg in saline in a volume <200 μl).

Within one to two weeks following RO administration of adriamycin, the mice exhibited cardiac dysfunction manifested by the following observations: reductions in left ventricular posterior wall (LVPW) thickening; or increases in end systolic volume (indicative of reduced contractility); reductions in end diastolic volume (indicative of cardiac remodeling and constrictive cardiomyopathy); reduced ejection fraction and or reduced cardiac output (indicative of diminished global cardiac function); increases in the sphericity index (calculated by left ventricular internal dimensions at end diastole divided by left ventricular long axis length at end diastole, indicating a more globular ventricular shape); and reductions in the slope of the early relaxation phase (indicative of constrictive cardiomyopathy and diminished elasticity of the ventricle). The early constrictive phase is subsequently followed by a chronic phase of continued reductions in contractility accompanied by ultimate cardiac dilation, evidenced by long term increases in end diastolic volumes.

Echocardiograms were performed after the administration of adriamycin to monitor the development and progression of cardiomyopathy. Control (untreated) mice developed substantial cardiac dysfunction within 5-7 days after the high dose adriamycin, with significant mortality 7-14 days after adriamycin challenge. Control (untreated) mice developed substantial cardiac dysfunction within two weeks after low dose adriamycin, with minimal mortality observed.

The changes observed in end diastolic volume and sphericity can be attenuated by continuous oral treatment with the beta 1-selective adrenergic receptor antagonist, metoprolol, at 100 mg/kg/day in the drinking water, resulting in what is termed “reverse remodeling” and enhanced cardiac output. Metoprolol is used in the clinical treatment of congestive heart failure and post-myocardial infarction cardiac dysfunction, and was used as a positive control in this model. Metoprolol's improvements in cardiac output (CO) are due predominantly to improved stroke volume (SV). CO is comprised of both SV and heart rate (HR).

C-Kit-+ cells reduced mortality from 80% to 20% and improved cardiac function in the high dose adriamycin-treated mice. C-Kit-+ cells enhanced cardiac contractility, limited changes in chamber dimensions, and improved global cardiac output in both high and low dose adriamycin-treated mice.

At the conclusion of the study, the vasculature was flushed in situ with cardioplegic solution until all blood had been removed and the heart arrested in diastole. The heart, spleen, liver, kidney, GI tract, and other organs were removed and either fixed in formalin, zinc formalin, or frozen for subsequent immunohistochemical analysis. Immunohistochemical analyses demonstrated the presence of DiI or BrdU positive cells within the heart, which had differentiated into cardiomyocytes, endothelial cells, smooth muscle cells, and cardiac interstitial cells.

### Example 3

### Role of C-Kit+ Cells in Tissue Repair in a Ligation Model of Ischemic Cardiomyopathy

Additionally, to determine the effect of cardiac c-Kit-+ cells in tissue repair, another mouse model of ischemic cardiomyopathy is used. In this model, the left anterior descending artery is ligated to induce myocardial ischemia.

C-Kit-+ cells are isolated from the heart or femur and sternum bone marrow of mice (as set out above in Example 1), expanded in vitro for three weeks, and injected either RO or IPC at a volume of 10,000 to 1,000,000 cells per mouse, three days after ligation or ischemia-reperfusion of the left anterior descending artery.

Mice (8-12 weeks old; typically five per experiment) are anesthetized by intraperitoneal injection of Avertin (20 mg/ml administered at 0.3-0.5 ml/mouse; 0.4-0.6 mg/gm). Animal's necks and chests are shaved and cleaned with alternating betadine and ethanol (70%). Animals are placed in a supine position on a platform with gauze padded rubberbands used to immobilized their paws. Their necks are extended by tauting ligature behind the front lower incisor. Body temperature is maintained throughout the entire procedure and during recovery by the use of circulating heat pumps.

Tracheotomy and intubation are performed as follows. After making a midline cervical skin incision, the trachea is isolated by separation of overlying muscle to allow visualization of intubation. Intubation is carried out by slightly retracting the tongue and inserting the beveled end of a PE-90 endotracheal tube through the larynx and into the trachea (5-8 mm). Mice are ventilated by the use of a Minivent mouse ventilator (Hugo Sach Elektroniks Model 845, D-7932) connected to the PE-90 endotracheal tube with a PE-160 tube. Room air is provided through an inspiration tube at 200 respirations/minute with a 200 μl stroke volume. A thoracotomy is then performed by a left midstemal skin incision at the 4-5 intercostal space (5-8 mm). The muscles overlying the intercostal space are gently separated and retracted by ligature to the front right incisor. A cut is then made through the intercostal space and the chest is opened using an intercostals retractor. The heart is accessed through the open site and it is right-laterally oriented. LAD occlusion is then accomplished by ligation of LAD by tying 8-0 suture around the artery. The suture is passed under the artery at a site 1 mm from the tip of the branching point.

C-Kit-+ cells, isolated fresh from mouse heart, marrow, liver, or other organs, or cultured c-Kit-+ cells maintained in IMDM with 2-10% horse serum, are labeled with a tracking dye such as DiI or BrdU. Labeled c-Kit-+ cells are injected either retroorbitally (RO) into the RO sinus or intrapericardially (IPC) into the pericardial sac into mice under isolflourane anesthesia at a volume of 10,000 to 1,000,000 cells per mouse, at least three days after the LAD is ligated.

Echocardiograms are performed after LAD ligation administration to monitor the development and progression of cardiomyopathy and the effect of c-Kit+ cells on tissue repair.

At the conclusion of the study, the vasculature is flushed in situ with cardioplegic solution until all blood had been removed and the heart arrested in diastole. The heart, spleen, liver, kidney, GI tract, and other organs are removed and either fixed in formalin, zinc formalin, or frozen for subsequent immunohistochemical analysis to determine the presence of DiI or BrdU positive cells within the heart.

### Example 4

### The Use of Embryoid Body-Like Cell Clusters Derived from C-Kit+Cells in a High Throughput Screening Assay

To determine the effect of different agents on differentiation and proliferation cardiac c-Kit-+ cells are isolated from the blood, bone marrow, tissue, heart or other organ, grown in culture to produce embryoid body-like cell clusters (EBLC), and used in subsequent screening assays. EBLC, which develop in vitro following culture of primary isolated c-Kit-+ cells, are dispersed into a single cell suspension using a brief one minute trypsin treatment. Trypsin digestion is stopped by the addition of IDMD containing 10% FBS. Individual cells are then replated into several different differentiation medias and used to identify factors which result in the appearance of lineage committed or fully differentiated cells of the following lineages: cardiomyocytes, endothelial cells, osteoblasts, chondrocytes, neurons, oligodendrocytes, adipocytes, smooth muscle cells, hematopoietic cells, hepatocytes, fibroblasts, renal cells, or germ cells. Differentiation media contain mixtures of growth factors and other agents chosen from among, but not limited to, the following growth factors and agents: indomethacin, insulin, epidermal growth factor, basic fibroblast growth factor, ascorbic acid, beta-glycerophosphate, thyroxine, tri-iodothyronine, dexamethosaone, 5-azacytidine, retinoic acid, DMSA, heparin sulfate, isobutylmethylxanthine, bone morphogenetic proteins, transforming growth factor beta, insulin growth factor, and parathyroid hormone.

Factors which are found to induce differentiation of the EBLC toward specific lineage pathways can be used to treat diseases and disorders such as: congestive heart failure, cardiomyopathy, angina, myocardial infarction, sudden cardiac death, peripheral vascular disease, rheumatoid arthritis or osteoarthritis, non-healing fractures, macular degeneration, stroke, Alzheimer's disease, Parkinson's disease and other neurodegenerative diseases.

Other agents can be used with the EBLC of the present invention in an embryonic stem cell test to determine their embryotoxicity or cytotoxicity (Laschinski et al., supra; Spielmann et al., supra; and Scholz et al., supra).

### Example 5

### The Use of Embryoid Body-Like Cell Clusters Derived from C-Kit+ Cells in the Treatment of Cardiomyopathy and Other Diseases

C-Kit-+ cells are isolated from the blood, bone marrow, tissue, heart or other organ, after treatment with G-CSF. C-Kit-+ cells are also isolated without G-CSF pretreatment. Cardiac c-Kit-+ cells are isolated from cardiac tissue biopsies, surgical explants or total explanted human hearts following the methods described in Example 1. The isolated c-Kit-+ cells are placed in long term culture until the appearance of EBLC is observed. The resulting EBLC are dispersed by gentle trypsin or versene digestion to single cell suspensions and then administered into the patient.

For patients with reduced cardiac function caused by any etiology, the administration may be by direct injection into the myocardial wall, by intra-coronary infusion catheter, or by intravenous injection to a recipient. The single cell suspension derived from the EBLC can differentiate into all cell types found in the heart, and in the appropriate in vivo milieu can be driven by local growth factors and other local cues to selectively replace or augment the specific cardiac cell types necessary for improvement of cardiac function in that specific patient.

### Example 6

### Isolation of Cardiac C-Kit+ Cells from Mice

To obtain cardiac c-Kit-+ cells, adult mouse hearts were digested and cardiomyocyte progenitor cells (cardiac c-Kit-+ cells) were isolated using a Worthington Neonatal Cardiomyocyte Isolation System (Taconic, Rensselaer, N.Y.; Cat. No. 33K6693) using conditions as set out below. This method appeared to be superior to a tissue digestion method using versene, collagenase type II, hyaluronidase IV-S, DNase I, and dispase, although both methods may be used.

The cardiomyocyte isolation kit contains sufficient materials for five separate tissue dissociations, each containing up to twelve hearts. For larger or smaller tissue samples prepare proportionate volumes of reagents at each step and combine them in the same ratio as described in the protocol. The contents of the kit are as follows:

Vial 1: 1 bottle, 500 ml: Sterile calcium- and magnesium-free Hank's Balanced Salt Solution (CMF HBSS), pH 7.4. The solution is used for reconstituting the contents of Vials #2 and #3 in addition to serving as the medium for the dissociation.

Vial 2: 5 vials, 1000 μg each: Worthington Trypsin (Code: TRLS), 3× crystallized, dialyzed against 1 mM HCl, filtered through 0.22 micron pore size membrane, and lyophilized. Before use, reconstitute with 2 ml CMF HBSS (Vial #1) and swirl gently to dissolve contents. Store at 2-8° C.

Vial 3: 5 vials, 2000 μg each: Worthington Soybean Trypsin Inhibitor (Code: SIC), a 0.22 micron pore size membrane filtered, lyophilized powder. Before use, reconstitute with 1 ml CMF HBSS (Vial #1) and swirl gently to dissolve contents.

Vial 4: 5 vials, 1500 Units each: Worthington Purified Collagenase (Code: CLSPA), a 0.22 micron pore size membrane filtered, lyophilized powder which has been chromatographically purified. It contains less than 50 caseinase units per milligram and is composed of two separable but very similar collagenases. Before use, reconstitute with 5 ml Leibovitz L-15 media (prepared as described below) and swirl gently to dissolve contents. Store at 2-8° C.

Pouch containing Leibovitz L-15 Media Powder: 1×1L, Reconstitute entire contents of pouch by cutting open top of envelope and pouring contents into beaker containing 800 ml of cell culture grade water. Rinse pouch 2-3 times with additional 100 ml. Bring total volume to 1 liter and filter through a 0.22 micron pore size filter.

The kit also includes 5 Cell Strainers (Falcon), a card correlating phenol red color with pH for checking balanced salt solutions and culture media.

The method was performed as follows: On the afternoon of day 1, heparin (50 units/100 μl total volume) was injected (IP) into 8-week old mice (C57B1/6, Taconic, Germantown, N.Y.) to aid in the flushing of blood out of the heart. Five adult mouse hearts were used per kit. Reagents used were: CMF HBSS: 50-60 ml from Vial #1, ice cold; Trypsin: reconstituted one Vial (#2) with 2 ml Reagent #1, ice cold; one sterile 50 ml centrifuge tube, in ice; and 10 cm Petri dish, sterile, on ice. The procedure was carried out as follows: Transfer 30-40 ml of Reagent #1 to the centrifuge tube. IP inject 300 μl Avertin into mice to anesthetize. Spray 70% EtOH on fur; trim away skin; open abdominal cavity; push liver aside to access diaphragm; and carefully snip away diaphragm to expose chest cavity, being cautious not to touch heart. Cut through ribs on both sides of rib cage and pull back sternum to expose heart. Open the abdominal cavity and push aside the intestines. Snip the abdominal aorta behind the intestines and let mouse begin to bleed out. Use a 27 gauge needle to cannulate the thoracic vena cava, and slowly inject 5 mls PBS containing 5 units heparin per ml. Perfusion of 5 mls PBS-heparin should take place at a rate of about 1 ml per minute to keep the pressure low. Heart will begin to blanch. The heart should remain beating during the cannulation, which will help to flush. Retrograde perfuse with another 5 mls PBS-heparin slowly for 5 minutes through the thoracic aorta. Carefully remove heart from chest cavity. Immediately place the hearts in the centrifuge tube containing Reagent #1 to chill and rinse. Repeat for remaining hearts. Swirl the tube to rinse hearts; then pour off most of the liquid. Rinse the hearts with 10 ml of Reagent #1; pour off the liquid as before; then transfer the hearts to the Petri dish. Mince the tissue with small scissors or a razor blade to less than 1 mm³ pieces keeping tissue at 0° C. Add Reagent #1 to Petri dish to a final volume of approximately 9 ml. Transfer 1 ml of the contents of the trypsin vial (Vial #2) into the Petri dish and mix completely by swirling. Final trypsin concentration is 50 μg/ml. Place the lid on the Petri dish and immediately place in refrigerator overnight (16-20 hours) at 2-8° C. shaking on rocker. Note: If animals are 4 days old or older, increase the trypsin concentration up to a maximum of 100 μg/ml.

On the morning of day 2, turn on 37° C. water bath; pre-chill two centrifuges; remove Ads buffer from freezer and thaw at 37° C. (1× Ads buffer comprises NaCl (6.8 g/L), KCl (0.4 g/L), Dextrose, 1.0 g/L, NaH₂PO₄ (1.5 g/L), HEPES (4.76 g/L), and MgSO₄ (0.1 g/L). Once thawed, put 1× Ads buffer on ice and 10× Ads buffer at room temperature (RT). Prepare: Reagent #1, CMF HBSS: 30 ml. ice cold; Reagent #3, Trypsin Inhibitor: reconstitute one of Vial #3 with 1 ml Reagent #1 at RT. Reagent #4, Collagenase: reconstitute one of Vial #4 with 5 ml prepared Leibovitz L-15 at RT. Prepare enough culture medium containing calcium and magnesium for digestion, centrifugations, and plating in cultureware. (approximately 100 ml for 10 hearts) at RT. Prepare wide-mouth 10 ml serological pipets, sterile (opening about 3 mm diameter; 25 ml pipet which had a large bore); and standard 10 ml plastic serological pipet. Remove Petri dish from refrigerator and bring to sterile hood on ice. Transfer tissue and buffer to 50 ml centrifuge tube on ice using wide-mouth pipet. Transfer contents of Vial #3 into tube and mix. Oxygenate tissue for 30 seconds to 1 minute if O² is available by passing oxygen over the surface of the liquid. Alternately, if O² is not available, gently pipet up and down. Warm tissue and buffer to 30-37° C. in water bath, maintaining sterility (i.e. cap if needed). Do not add calcium-containing medium until tissue fragments are warm. Slowly transfer the contents of Vial #4 into tube and mix. Cap tube tightly. Place tube in shaking water bath at 37° C. and incubate for 30 to 45 minutes. All subsequent steps are performed at RT. Remove tube from incubator and return to sterile hood. With standard 10 ml plastic serological pipet, triturate about 10 times to release cells. (Trituration is discussed in the following inset.) Pipet as gently as possible, consistent with successful tissue dispersion. Rinse a cell strainer with 1 ml of the L-15 culture medium. Allow tissue residue to settle 3-4 minutes; then (with same pipet) filter the supernatant through the cell strainer into a fresh 50 ml centrifuge tube. Add 5 ml additional L-15 culture medium to tissue residue; repeat trituration step. Allow tissue residue to settle as before; then filter cells through the same cell strainer. Rinse mesh gently with 2 ml culture medium. Oxygenate cells for 1 minute; then allow filtered cells to remain undisturbed for about 20 minutes at room temperature. This allows complete digestion of the partially degraded collagen. (Cells can be held up to 1 hour at this point, make up Percoll gradient during this period.) Swirl cells gently; if no clumps have formed and appearance is uniform, sediment cells at 50-100×g for 5 minutes (enough to settle the myocytes and some but not all red cells.) Cells were spun down at 1220 rpm. With adequate flushing, there should be very few contaminating RBCs. At this stage, routine cell yields are 2-3×10⁶ cardiomyocytes per heart digested. Cells were not counted but went directly to a Percoll gradient.

Percoll purification was performed as follows: In a 50 ml conical tube, make stock solution using 9 parts (45 mls) Percoll plus 1 part (5 mls) 10× Ads without phenol red. Use Percoll stock and 1× Ads buffer to dilute Percoll solutions as shown in the following table (this will make 250 ml tube gradients).

| Percoll Density (g/ml) | Percoll Stock (ml) | 1× Ads (+/− phenol red) | |
|---|---|---|---|
| 1.050 | 9 | 16 | (−red) |
| 1.060 | 11.1 | 13.9 | (+red) |
| 1.082 | 15.8 | 9.2 | (−red) |

One Percoll gradient was used to separate cells; an extra Percoll gradient was used to act as a centrifuge balance.

Resuspend cell pellet in 1.082 g/ml Percoll. Prepare Percoll gradient: Add 12 mls of 1.050 density to each 50 ml conical tube. Underlay 12 mls of 1.060 density (red) Percoll. Next, carefully underlay 1.082 density containing the cells. Centrifuge at 3000 rpm at 4° C. for 30 minutes with no brake. Use a transfer pipette to carefully suction myocyte layer from the band. Resuspend cells in plating media, wash and centrifuge 2× at 1650 rpm for 5 min. The supernatant was saved and re-spun it at 3000 rpm to make sure all small cells had pelleted down. The supernatant and the pellet were then plated. The cell pellet was plated in 10 mls of DF media containing 5% horse serum for overnight culture. Media was changed the next day to DF containing no serum. Cells were then sorted using a fluorescence activated cell sorter (FACS) for cardiac c-Kit-+ cells.

C-Kit-+ cells are either selected immediately after Percoll gradient procedure or after one day in culture. C-Kit-+ cells are either FACS-sorted or selected by using an EASY SEP method (StemCell Technologies, Vancouver, BC) of positive selection. After heart tissue was digested as set out above, cell suspensions were run over a 70 micron filter and then a 30 micron filter. Cells were then stained with c-Kit FITC antibody and incubated with an EASY SEP anti-FITC selection cocktail. Magnetic nanoparticles were added. Cells were placed into a magnet and the supernatant was poured off and cells were rinsed twice and supernatant was poured off each time. Cells were then collected by simply removing the tube from the magnet, because positive cells remained in the tube. Cells can then be cultured, injected into animals for therapeutic purposes, or analyzed further via FACS or immunohistochemistry.

Cell freezing: Cardiac c-Kit-+ cells, isolated from mouse hearts after enzyme digestion, magnetic cell sorting (MACS column isolation), and FACS sorting were grown in culture and fed approximately once per month with IMDM+FBS (10%), penicillin (100 units/ml), streptomycin (100 μg/ml), and glutamine (292 μg/ml) (IMDM/FBS/PSG). Cells were frozen at concentrations of 5 million cells/vial in 15% serum and 10 million cells/vial in 30% serum. Vials were placed in an insulated container at −70° C. and then transferred for long term storage in liquid nitrogen.

Observations: Immediately after plating, cells appeared mostly round but looked healthy. Some larger clumpy-looking myocytes were also present, as well as very few driftwood shaped myocytes. One hour after plating, some cells began to stick down on the plate, but maintained their round shape. Many cells were still floating at the one hour time point. Supernatant was removed from the flask and transferred to a fresh flask. Serum-free DF media is added to the attached cells in the first flask. Many fibroblasts appeared to have sat down; some unhealthy partially attached myocytes also existed. The attached cells in the first flask contained many fibroblasts. Some partially attached or floating myocytes were present. Some small round cells in suspension and some debris were also observed. The supernatant was removed to a new flask and fresh serum free DF medium was added to the first flask. Cells were incubated long term and monitored for changes in differentiation and/or proliferation.

### Example 7

### Embryoid Body-Like Cell Clusters Derived from Progenitor Cells in Human Umbilical Cord Blood

Progenitor cells (pluripotent human stem cells, which are capable of giving rise to multiple cell lineages), derived from human umbilical cord blood, also give rise in culture to EBLC, similar to EBLC that we have observed after culture of adult mouse cardiac-derived c-Kit-+ cells. EBLC were isolated and cultured from human umbilical cord blood as set out below.

Fresh human umbilical cord blood (40-60 ml) was anti-coagulated with Na heparin (100 U/ml) and stored at 4° C. for approximately 4-8 days to remove or deplete cells of committed lineages (non-stem cells) by reducing the number of viable differentiated lymphocytes (or by killing off differentiated non-progenitor cells) and preserve progenitor cell survival. [Cells of committed lineages (non-stem cells) are depleted by refrigeration for 4-8 days. Likewise, positive selection of stem cells (c-Kit+ cells) using FACS or MACS is an alternate method of removing non-stem cells.] Whole blood was then diluted 1:1 with PBS, without magnesium and calcium. Whole blood (WB)-PBS (35 ml) was layered onto 15 ml Isolymph (Gallard Schlesinger Industries) at RT and then spun at 2000 rpm at RT for 30 min in a centrifuge (Beckman GS-6R) with the brake off. The decant from the first washing (4 ml of mononuclear cell (MNC) band plus 45 ml PBS without calcium and magnesium) was saved. Red blood cells (RBCs) in the pellet were lysed with distilled water followed by 10× PBS. Both washes were spun at 2000 rpm at RT for 10 min in a centrifuge (Beckman GS-6R).

Total yield in several trials was approximately 5.0-8.0×10⁶ mononuclear cells (MNCs)/40-60 mls blood. Cells were plated in a 6-well plate at a concentration of 1.0×10 cells/2 ml of RPMI with 10% FBS and 1.0×10⁶ cells/2 ml of DMEM/F12 Ham's with 10% FBS. (The decant was also plated; however, less than 2% of the cells were viable by trypan blue exclusion.) Remaining cells (from the RBC lysis) were frozen at 2×10⁶ cells in RPMI with 20-30% FBS and 5-10% DMSO. Cells were grown for several days and culture media was added and changed. Cultures gave rise to both adherent and non-adherent cells, which are self-renewing but appear to require the presence of each other for survival, as neither cell type does well alone. Adherent cells also seemed to prefer scratched polystyrene surfaces on cultures plates, because cells attached and grew on scratches. Both adherent and non-adherent cells appear to give rise to non-adherent and adherent cells, respectively.

After culturing MNCs for approximately two weeks, single cell cloning was begun. The rationale is that by this time period, any remaining fully differentiated cells would have died off and only progenitor cells should be remaining. Both RPMI with 10% FBS and DF12 with 10% FBS was used to begin single cell cloning of cells in 96-well plates. Two days after cells were plated, plates were examined for single cell wells. Single cell wells were chosen and grown in either DF12+10% FBS or RPMI+10% FBS. After 33 days post-isolation, cells grown in DF12+10% FBS began attaching to the flask and spreading out. Cells in RPMI+10% FBS appeared to grow more slowly, but appeared to be both self-renewing and differentiating; thus, they appeared to be stem cells. (A cell which is a progenitor cell alone is not self-renewing, only differentiating.) EBLC formed in culture from isolated human mononuclear cells. EBLC, isolated by this method, can have many uses as discussed herein. EBLC can be used as a screening tool to identify factors that promote or inhibit differentiation or proliferation. EBLC can also be used in toxicity screening assays. EBLC may also be used in cell replacement therapy.

Stem cells, derived from digestion of EBLC, were then separated into several flasks with various types of growth media (all from Cambrex Bioscience (Walkersville, Md.): smooth muscle media (CC-3182), skeletal muscle media (CC-3160), astrocyte media (CC-3186), and endothelial media (CC-3156) to promote differentiation into various cell types, i.e., smooth muscle cells, skeletal muscle cells, astrocytes, and endothelial cells, respectively. Cells are cultured in various media to observe proliferation and differentiation. Cells are also stained immunohistochemically to detect the presence of various differentiation markers.

### Example 8

### Differentiation of Cells Derived from Human Umbilical Cord Blood into Cardiac-Specific Cells

To determine if stem cells could be differentiated into cardiac-specific progenitor cells in vitro, stem cells, derived from human umbilical cord vein as set out in Example 7, were co-cultured with irradiated rat myocytes as a feeder layer. A feeder layer of irradiated myocytes is used for the production of growth factors, which act to promote stem cell differentiation into cardiac-specific cells.

Rat myocytes were isolated and plated at 1.5-2.0×10⁶ cells/well in six-well plates. After four days of culture, myocytes were irradiated in a cesium irradiator at 764 rads/min, using 3000 rads (3.93 min at position 2). One hour after irradiation, myocytes were still alive and beating. Stem cells, derived from the culture and subsequent digestion of EBLC (obtained from a culture method as set out in Example 7) were then spun down and counted. Stem cells (5×10⁵ cells) were then plated onto each well of irradiated myocytes (1.5-2.0×10⁶ cells/well).

Cells are then co-cultured for several days through several months and examined for differentiation and proliferation. Cells are then analyzed for cardiac-specific phenotype and frozen for later use. Cells, identified as cardiac-specific progenitor cells, are then grown in culture for organ culture experiments, administered into a mammal for cell replacement therapy, or frozen for future experimental use.

Alternately, stem cells are cultured with 5-azacytidine [pulsed with 5-azacytidine at a concentration of 10 nm-100 μm (preferred concentration ranges from 5-10 μm) for 24-48 hours] to induce cardiocyte differentiation. Stem cells are also induced to differentiate into cardiocytes by treatment with insulin-like growth factor (IGF), HGF, or agents which modulate the Wnt pathway, i.e., Dickkopf (Dkk). Dkk is a negative regulator of Wnt signaling. Additional agents, which induce cardiocyte differentiation, are known to one of skill in the art and are also used to analyzed stem cell differentiation.

### Example 9

### Embryoid Body-Like Cell Clusters Derived from Progenitor Cells in Placenta

Stem cells are also derived from placental progenitor cells and give rise in culture to EBLC. A method for isolating and culturing EBLC from placental progenitor cells is set out below.

Human placental MNCs are isolated from trypsin-digested term placentas. (See Fukuchi et al., Stem Cells 22:649-58, 2004, for a method of trypsin-digesting placental tissue and cell isolation). After cells are collected using trypsin digestion, cells (diluted with PBS, without magnesium and calcium) are stored at 4° C. for approximately 4-8 days to reduce the number of viable differentiated lymphocytes (or to kill off differentiated non-progenitor cells) and preserve progenitor cell survival. Cells in PBS (35 ml) are layered onto 15 ml Isolymph (Gallard Schlesinger Industries) at RT and then spun at 2000 rpm at RT for 30 min in a centrifuge (Beckman GS-6R) with the brake off. The decant from the first washing (4 ml of mononuclear cell (MNC) band plus 45 ml PBS without calcium and magnesium) is saved. Red blood cells (RBCs) in the pellet were lysed with distilled water followed by 10× PBS. Both washes are spun at 2000 rpm at RT for 10 min in a centrifuge (Beckman GS-6R). Cells are then plated and cultured, as set out in Example 7, for the development of EBLC. Likewise, cells are frozen for later use. Placental progenitor cells are useful in cell replacement therapy and as screening tools in assays which identify differentiation, proliferation, or toxicity factors.

1. A method for producing embryoid body-like cell clusters derived from c-Kit-+ cells from a mammal, wherein the method comprises:
a.) administering to a mammal a composition comprising an effective amount of granulocyte colony stimulating factor (G-C SF) polypeptide to mobilize c-Kit+ cells;
b.) isolating the mobilized c-Kit+ cells from the blood, bone marrow, tissue heart or other organ; and
c.) culturing the c-Kit+ cells in long-term culture medium for the development of the embryoid body-like cell clusters.

2. The method of claim 1 wherein the composition includes at least one additional growth factor.

3. The method of claim 2 wherein the growth factor is stromal derived growth factor-1 (SDF-1).

4. In an improved method of cell replacement therapy, the improvement comprising:
a.) administering to a mammal a composition comprising an effective amount of granulocyte colony stimulating factor (G-C SF) polypeptide to mobilize c-Kit+ cells;
b.) isolating the mobilized c-Kit-+ cells from the blood, bone marrow, tissue, heart or other organ;
c.) culturing the c-Kit+ cells in long-term culture medium for the development of the embryoid body-like cell clusters; and
d.) administering the embryoid body-like cell clusters into a mammal for cell replacement therapy.

5. The method of claim 4, wherein the cell replacement therapy is used in the treatment of various diseases or disorders.

6. The method of claim 5, wherein the disease or disorder is selected from the group consisting of: cardiac myopathies, skeletal myopathies, neural degenerative diseases, stroke, bone loss, vascular degeneration, and joint degeneration.

7. The method of claim 4 wherein the composition includes at least one additional growth factor.

8. The method of claim 7 wherein the growth factor is stromal derived growth factor-1 (SDF-1).

9. In an improved method for screening agents for their ability to promote cell differentiation, the improvement comprising:
a.) administering to a mammal a composition comprising an effective amount of granulocyte colony stimulating factor (G-CSF) polypeptide to mobilize c-Kit-+ cells;
b.) isolating the mobilized c-Kit-+ cells from the blood, bone marrow, tissue, heart or other organ;
c.) culturing the c-Kit-+ cells in long-term culture medium for the development of the embryoid body-like cell clusters;
d.) treating the embryoid body-like cell clusters with agents; and
e.) examining the embryoid body-like cell clusters for changes in cellular phenotype.

10. The method of claim 9, wherein the cellular phenotype is selected from the group consisting of: cardiomyocytes, endothelial cells, osteoblasts, chondrocytes, neurons, oligodendrocytes, adipocytes, smooth muscle cells, hematopoietic cells, hepatocytes, fibroblasts, renal cells, and germ cells.

11. The method of claim 9, wherein the agent is derived from a chemical, protein, or other library.

12. The method of claim 9 wherein the composition includes at least one additional growth factor.

13. The method of claim 12 wherein the growth factor is stromal derived growth factor-1 (SDF-1).

14. In an improved method for screening agents for toxicity, the improvement comprising:
a.) administering to a mammal a composition comprising an effective amount of granulocyte colony stimulating factor (G-CSF) polypeptide to mobilize c-Kit+cells;
b.) isolating the mobilized c-Kit-+ cells from the blood, bone marrow, tissue, heart or other organ;
c.) culturing the c-Kit-+ cells in long-term culture medium for the development of the embryoid body-like cell clusters;
d.) treating the embryoid body-like cell clusters with agents that affect cell differentiation and proliferation; and
e.) examining the embryoid body-like cell clusters for changes in cellular phenotype or cell number.

15. A method for producing embryoid body-like cell clusters derived from c-Kit-+ cells from a mammal, wherein the method comprises:
a.) isolating the c-Kit-+ cells from the blood, bone marrow, tissue heart or other organ; and
b.) culturing the c-Kit-+ cells in long-term culture medium for the development of the embryoid body-like cell clusters.

16. In an improved method of cell replacement therapy, the improvement comprising:
a.) isolating c-Kit-+ cells from the blood, bone marrow, tissue, heart or other organ;
b.) culturing the c-Kit-+ cells in long-term culture medium for the development of the embryoid body-like cell clusters; and
c.) administering the embryoid body-like cell clusters into a mammal for cell replacement therapy.

17. The method of claim 16, wherein the cell replacement therapy is used in the treatment of various diseases or disorders.

18. The method of claim 17, wherein the disease or disorder is selected from the group consisting of: cardiac myopathies, skeletal myopathies, neural degenerative diseases, stroke, bone loss, vascular degeneration, and joint degeneration.

19. In an improved method for screening agents for their ability to promote cell differentiation, the improvement comprising:
a.) isolating c-Kit-+ cells from the blood, bone marrow, tissue, heart or other organ;
b.) culturing the c-Kit-+ cells in long-term culture medium for the development of the embryoid body-like cell clusters;
c.) treating the embryoid body-like cell clusters with agents; and
d.) examining the embryoid body-like cell clusters for changes in cellular phenotype.

20. The method of claim 19, wherein the cellular phenotype is selected from the group consisting of: cardiomyocytes, endothelial cells, osteoblasts, chondrocytes, neurons, oligodendrocytes, adipocytes, smooth muscle cells, hematopoietic cells, hepatocytes, fibroblasts, renal cells, and germ cells.

21. The method of claim 19, wherein the agent is derived from a chemical, protein, or other library.

22. In an improved method for screening agents for toxicity, the improvement comprising:
a.) isolating c-Kit-+ cells from the blood, bone marrow, tissue, heart or other organ;
b.) culturing the c-Kit-+ cells in long-term culture medium for the development of the embryoid body-like cell clusters;
c.) treating the embryoid body-like cell clusters with agents that affect cell differentiation and proliferation; and
d.) examining the embryoid body-like cell clusters for changes in cellular phenotype or cell number.

23. A method for producing embryoid body-like cell clusters derived from umbilical cord blood stem cells from a mammal, wherein the method comprises:
a.) isolating mononuclear cells from umbilical cord blood;
b.) removing non-stem cells from the mononuclear cells; and
c.) culturing the remaining mononuclear cells in long-term culture medium for the development of the embryoid body-like cell clusters.

24. A method for producing embryoid body-like cell clusters derived from placental stem cells from a mammal, wherein the method comprises:
a.) isolating mononuclear cells from placenta;
b.) removing non-stem cells from the mononuclear cells; and
c.) culturing the remaining mononuclear cells in long-term culture medium for the development of the embryoid body-like cell clusters.

25. A method for cell replacement therapy comprising administering to a mammal an effective amount of the embryoid body-like cell clusters according to claim 23 or 24.

26. A method for screening agents for their ability to promote stem cell differentiation comprising:
a.) administering an agent to an embryoid body-like cell cluster according to claim 23 or 24, and
b.) examining the embryoid body-like cell cluster for changes in cellular phenotype.

27. A method for screening agents for their ability to promote or inhibit cardiocyte differentiation comprising:
a.) administering an agent to an embryoid body-like cell cluster according to claim 23 or 24, and
b.) examining the embryoid body-like cell cluster for expression of a cardiocyte differentiation marker.

28. A method for screening agents for their ability to promote or inhibit stem cell proliferation comprising:
a.) administering an agent to an embryoid body-like cell cluster according to claim 23 or 24, and
b.) examining the embryoid body-like cell cluster for changes in cellular proliferation.

29. A method for producing cardiac progenitor cells derived from umbilical cord blood stem cells from a mammal, wherein the method comprises:
a.) isolating mononuclear cells from umbilical cord blood;
b.) removing non-stem cells from the mononuclear cells;
c.) culturing the remaining mononuclear cells in long-term culture medium for the development of embryoid body-like cell clusters;
d.) co-culturing the embryoid body-like cell cluster with irradiated myocytes or other agent which promotes cardiac progenitor cell differentiation;
e.) analyzing the embryoid body-like cells for the presence of cardiocyte differentiation markers; and
f.) selecting embryoid body-like cells of a cardiocyte phenotype.

30. A method for cell replacement therapy comprising administering to a mammal an effective amount of cells of a cardiocyte phenotype according to claim 29.

31. The method of any of claims 23, 24, and 29, wherein the removal of non-stem cells is carried out by refrigerating the cells.

32. The method of any of claims 23, 24, and 29, wherein the removal of non-stem cells is carried out by selecting c-Kit-+ cells.

33. The method of claim 33, wherein c-Kit-+ cells are selected by using a fluorescence activated cell sorter (FACS) or magnetic cell sorting (MACS).
